(19) United States
(12) Patent Application Publication
(10) Pub. No.: US 2013/0317557 A1
(43) Pub. Date: Nov. 28, 2013

(54) MIS ROD INSERTION DEVICE AND METHOD

(71) Applicant: CUSTOM SPINE, INC., Parsippany, NJ (US)

(72) Inventor: Mahmoud F. Abdelgany, Rockaway, NJ (US)

(73) Assignee: CUSTOM SPINE, INC., Parsippany, NJ (US)

(21) Appl. No.: 13/888,545

(22) Filed: May 7, 2013

Related U.S. Application Data
(60) Provisional application No. 61/652,172, filed on May 26, 2012.

(51) Int. Cl. A61B 17/70

(52) U.S. Cl. CPC A61B 17/7074
USPC 606/86 A

(57) ABSTRACT

A medical device includes an implant portion; an inserter portion detachably connected to the implant portion; and a device manipulation portion operatively connected to the inserter portion. In one embodiment, the implant portion includes a longitudinal member. The device may further include a breakaway component in between the implant portion and the inserter portion. The device may further include a notch configured between the implant portion and the inserter portion. The implant portion and the inserter portion are detachable upon applying a sufficient force to the device manipulation portion to cause detachment of the implant portion from the inserter portion. The detachment of the implant portion from the inserter portion occurs after the implant portion is seated in a construct assembly that is attached to a bone inside a patient.

### CROSS-REFERENCE TO RELATED APPLICATIONS

This application claims priority to U.S. Provisional Application Ser. No. 61/652,172 filed on May 26, 2012, the complete disclosure of which, in its entirety, is herein incorporated by reference.

### BACKGROUND

1. Technical Field

The embodiments herein generally relate to medical devices, and, more particularly, to medical devices used for spinal implant surgeries.

2. Description of the Related Art

Conventional spinal rods are typically inserted into a pedicle screw assembly using a separate rod insertion device. The surgeon typically holds the rods using the insertion device and upon setting the rod in place simply removes the insertion device from inside the patient. The insertion device is typically configured to be re-used for subsequent surgeries. U.S. Pat. Nos. 5,989,255; 6,193,719; 6,478,795; and 7,967,821 along with U.S. Patent Application Publication No. 2009/0228052, the complete disclosures of which, in their entireties, are herein incorporated by reference, teach various technologies related to screws, blockers, and pins.

### SUMMARY

In view of the foregoing, an embodiment herein provides a monolithic breakaway rod where the inserter portion is part of the rod and breaks off once insertion is complete. The embodiments herein include a medical device comprising an implant portion; an inserter portion detachably connected to the implant portion; and a device manipulation portion operatively connected to the inserter portion. In one embodiment, the implant portion comprises a longitudinal member. The device may further comprise a breakaway component in between the implant portion and the inserter portion. The device may further comprise a notch configured between the implant portion and the inserter portion. The implant portion and the inserter portion are detachable upon applying a sufficient force to the device manipulation portion to cause detachment of the implant portion from the inserter portion. The detachment of the implant portion from the inserter portion occurs after the implant portion is seated in a construct assembly that is attached to a bone inside a patient.

The breakaway component may comprise a bar having a thickness less than or equal to a thickness of any of the implant portion and the inserter portion. A portion of the breakaway component remains attached to any of the implant portion and the inserter portion upon detachment of the implant portion from the inserter portion. The device manipulation portion may comprise a handle. In one embodiment, the inserter portion comprises a thickness less than or equal to a thickness of the implant portion. The notch may be positioned on at least one side of the breakaway component. In one embodiment, the breakaway component comprises a uniform thickness. In another embodiment, the breakaway component comprises a tapered configuration. The device may further comprise a first angled portion in between the implant portion and the inserter portion; and a second angled portion in between the inserter portion and the device manipulation portion. Additionally, the device may further comprise at least one angled portion in the device manipulation portion.

Another embodiment provides a method of inserting an implant in a patient, the method comprising placing a pedicle screw construct assembly in the patient; positioning an implant portion of a medical device into the pedicle screw construct assembly using an inserter portion that is detachably connected to the implant portion; seating the implant portion into the pedicle screw construct assembly; forceably detaching the implant portion from the inserter portion; and removing the inserter portion from the patient. The method may further comprise manipulating the inserter portion with a handle. The implant portion and the inserter portion are detachable upon applying a sufficient force to the handle to cause detachment of the implant portion from the inserter portion. The forceably detaching of the implant portion from the inserter portion comprises any of pulling, pushing, bending, and twisting the inserter portion with respect to the implant portion. The method may further comprise discarding the insertion portion after removing the inserter portion from the patient.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A illustrates a top view of a medical device according to an embodiment herein;

FIG. 1B illustrates a perspective view of the medical device of FIG. 1A according to an embodiment herein;

FIG. 2 illustrates a magnified perspective view of the notch portion of the medical device of FIGS. 1A and 1B according to an embodiment herein;

FIG. 3A illustrates a cross-sectional view of an example notch portion according to a first embodiment herein;

FIG. 3B illustrates a top view of the notch portion of FIG. 3A according to a first embodiment herein;

FIG. 3C illustrates a side view of the notch portion of FIG. 3A according to a first embodiment herein;

FIG. 4A illustrates a cross-sectional view of an example notch portion according to a second embodiment herein;

FIG. 4B illustrates a top view of the notch portion of FIG. 4A according to a second embodiment herein;

FIG. 4C illustrates a side view of the notch portion of FIG. 4A according to a second embodiment herein;

FIG. 5A illustrates a cross-sectional view of an example notch portion according to a third embodiment herein;

FIG. 5B illustrates a top view of the notch portion of FIG. 5A according to a third embodiment herein;

FIG. 5C illustrates a side view of the notch portion of FIG. 5A according to a third embodiment herein;

FIG. 6 illustrates a top view of the medical device of FIGS. 1A and 1B with an attached handle according to an embodiment herein; and

FIG. 7 is a flow diagram illustrating a method according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

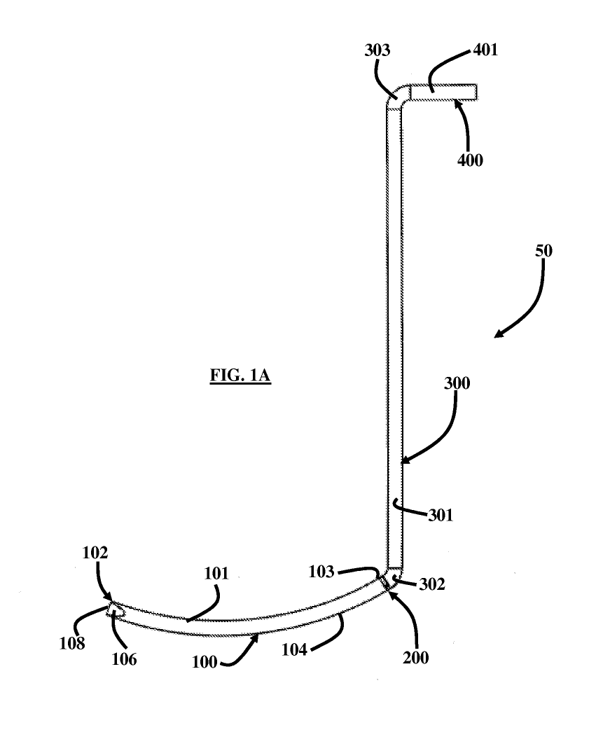

The embodiments herein provide a monolithic breakaway rod, wherein the inserter portion is initially integral to the rod, and the inserter portion breaks off once the insertion of the rod is completed. Referring now to the drawings, and more particularly to FIGS. 1A through 7, where similar reference characters denote corresponding features consistently throughout the figures, there are shown preferred embodiments.

As shown in FIGS. 1A and 1B, the medical device 50 provided by the embodiments herein comprises an implant portion 100, a notch 200 operatively connected to the implant portion 100, an inserter portion 300 operatively connected to the implant portion 100 by way of a notch 200 and breakaway bar 201, and a manipulation portion 400 operatively connected to the inserter portion 300.

The implant portion 100 may be embodied as a longitudinal member 101 having any of a curved, straight, round, and non-round configuration. The longitudinal member 101 may be shaped like a rod with curved surfaces 104 to permit seating in a complimentary screw head of a typical pedicle screw assembly (not shown) such as that described in U.S. Pat. No. 7,862,594, for example, the complete disclosure of which, in its entirety, is herein incorporated by reference, or any other pedicle screw assembly used in the industry which accommodates a longitudinal member such as a rod, and the embodiments herein are not limited to any particular screw head or pedicle screw assembly configuration.

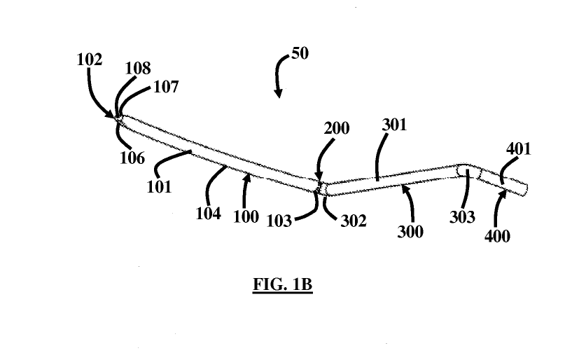

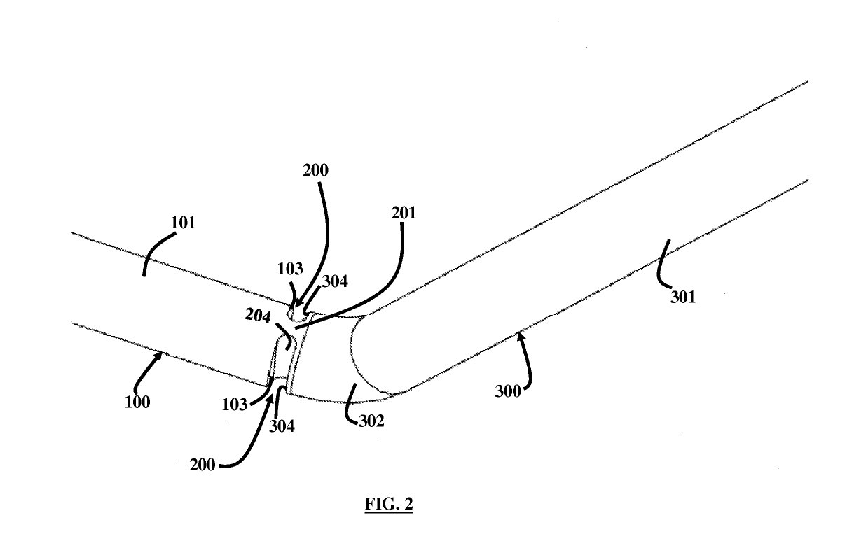

The longitudinal member 101 may comprise a tapered end 102 in one embodiment. The tapered end 102 may comprise a pair of sloping walls 106, 107 converging together in a cap area 108. A second end 103 may be configured at a distally opposite end from the tapered end 102. The description of the second end 103 is further described below with respect to FIG. 2. While the descriptions and drawings herein refer to a longitudinal member 101 being rod-like in shape, the embodiments herein are not restricted to this particular configuration as other shapes and geometries of the longitudinal member 101 may be configured in accordance with the embodiments herein. Moreover, the implant portion 100 may be embodied in non-longitudinal configurations including wedges or other shapes and geometries associated with a spinal implant or other surgical implant medical devices. Regardless of the configuration, the implant portion 100 is adapted to remain inside the patient (not shown) after the construct assembly (e.g., pedicle screw assembly attached to the bone of a patient) (not shown) has taken place and insertion of the implant portion 100 into the construct assembly is completed.

The notch 200 is positioned between the implant portion 100 and the inserter portion 300. The notch 200 is adapted to facilitate breakaway of the implant portion 100 from the inserter portion 300 (and attached manipulation portion 400) after the construct assembly (not shown) is completed. The configuration of the notch 200 may be selected from any appropriate shape or geometry such that the notch 200 may be smooth, jagged, curved, straight-angled, etc. so long as breakaway of the implant portion 100 can be facilitated by the user (i.e., surgeon) of the device 50. Furthermore, the configuration of the notch 200 is provided to facilitate stability of the implant portion 100 while it is connected to the inserter portion 300, while also allowing for minimal detachment force or torque required for breakaway of the implant portion 100 from the inserter portion 300 after the construct assembly has been completed and the user (i.e., surgeon) is ready to detach the inserter portion 300 from the implant portion 100.

Several factors may contribute to the amount of force or torque required to detach implant portion 100 from the inserter portion 300. These factors include, but are not limited to the type of material and diameter of the implant portion 100, as well as the type of material and size of the breakaway bar 201, as further described with respect to FIG. 2. Preferably, the notch 200 and breakaway bar 201 comprise geometric and material property characteristics that avoid excessively sharp edges from forming after breakaway of the implant portion 100 occurs.

FIG. 2, with reference to FIGS. 1A and 1B, illustrates a close-up view of the notch 200 according to an embodiment herein. As shown, the notch 200 is configured on either side of the breakaway bar 201 according to an embodiment herein. However, in other embodiments, the notch 200 may be configured on only one side of the breakaway bar 201 or all around the breakaway bar 201. The second end 103 of the longitudinal member 101 forms one side of the notch 200. Another side of the notch 200 is formed by a breakaway end 304 of the inserter portion 300. The other side of the notch 200 is formed by a bar wall 204 of the breakaway bar 201. The bar wall 204 may be curved or straight-angled. The thickness of the breakaway bar 201 is dependent on the overall thickness/diameter of the longitudinal member 101 and/or geometric characteristics and/or material property characteristics of the longitudinal member 101 and/or the breakaway bar 201.

As indicated in FIGS. 1A through 2, the inserter portion 300 is adjacent to the notch 200 and the manipulation portion 400. In one embodiment, the inserter portion 300 comprises a first elbow section 302 that is operatively connected to the breakaway bar 201. The breakaway end 304 of the first elbow section 302, which attaches to the breakaway bar 201, helps form the notch 200 as described above. The inserter portion 300 further includes an elongated member 301, which acts as an inserter rod for inserting the implant portion 100 into the construct assembly (e.g., pedicle screw assembly) (not shown). The length of the elongated member 301 is generally longer than the implant portion 100, and in one embodiment may be greater than twice the length of the implant portion 100. However, the relative size of the elongated member 301 compared with the size of the implant portion 100 is also dependent on the type of implant portion (e.g., longitudinal member 101 or some other type of implant) as well as the thickness/diameter and/or material property characteristics of the inserter portion 300, and the embodiments herein are not limited to a particular relative geometric configuration of the various components of the device 50. Furthermore, the inserter portion 300 also includes a second elbow section 303 located at a distally opposite end from the first elbow section 302 such that the elongated member 301 separates and is adjacent to each of the first elbow section 302 and second elbow section 303.

Preferably, the inserter portion 300 is disposable after construct assembly and the implant portion 100 has been broken away from the inserter portion 300. Moreover, the inserter portion 300 is configured to be of adequate and sufficient length and angle to allow easy insertion of the implant portion 100 onto and into the construct assembly (not shown). Preferably, the inserter portion 300 comprises a diameter and/or thickness that does not exceed the diameter and/or thickness of the implant portion 100 in order to facilitate minimally invasive surgery (MIS) insertion without (i) utilizing valuable space in the patient, (ii) reducing visibility of the user (i.e., surgeon) during performance of the operation, or (iii) causing excessive tissue disruption which could result in undesirable consequences such as injury or infection of the patient.

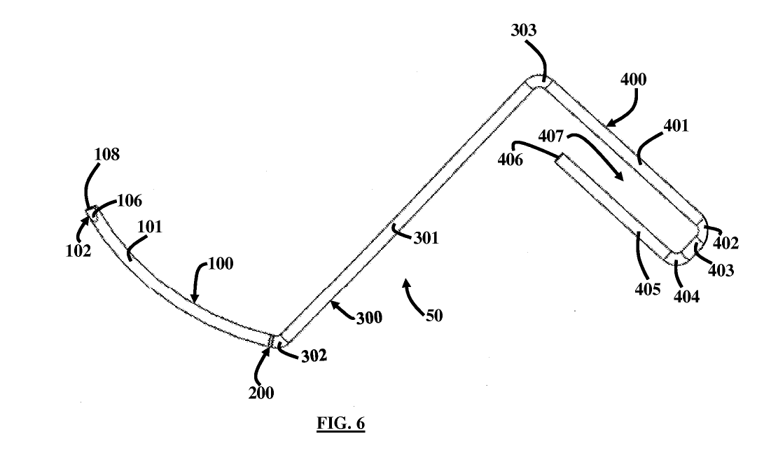

The manipulation portion 400 extends from the second elbow section 303 of the inserter portion 300 of the device 50. In one embodiment, the manipulation portion 400 may be a continuation of the second elbow section 303, and thus a continuation of the inserter portion 300 of the device 50. The manipulation portion 400 is adapted to facilitate comfortable insertion and separation of the implant portion 100 from the inserter portion 300. The manipulation portion 400 may be configured as a handle to permit a comfortable grip for the user (i.e., surgeon) to manipulate and use the device 50. In one embodiment, the manipulation portion may comprise an AO fitting (not shown) to accommodate a quick release handle (not shown). The handle shown in FIG. 6 is merely illustrative of just one kind of handle that may be configured, and the embodiments herein are not limited to one particular geometry, shape, or configuration. As shown in the example embodiment of FIG. 6, with reference to FIGS. 1A through 5, the manipulation portion 400 may comprise a first handle bar 401 attached to the second elbow section 303 of the inserter portion 300. A third elbow section 402 is operatively connected to the first handle bar 401. A connector 403 is positioned between the third elbow section 402 and a fourth elbow section 404. A second handle bar 405 is operatively connected to the fourth elbow section 404. The second handle bar 405 terminates at handle end 406. The length of the connector 403 creates a gap 407 between the first handle bar 401 and the second handle bar 405 to permit a user's (i.e., surgeon's) hand to fit therein.

The various elbow sections 302, 303, 402, and 404 may be angled and configured to permit the overall device 50 to have a specific configuration. For example, the angle of the first elbow section 302 allows the implant portion 100 to be angled with respect to the elongated member 301 of the inserter portion 300. Similarly, the angle of the second elbow section 303 allows the manipulation portion 400 to be angled with respect to the elongated member 301 of the inserter portion 300. The third and fourth elbow sections 402, 404 together with the connector 403 form the gap 407 between the first handle bar 401 and the second handle bar 405.

The various structures and components of the device 50 may be a single, monolithic, continuous, integrally-formed structure, or intermediate components (e.g., screws, hinges, glue, etc.) (not shown) may be used to operatively connect the various components together (e.g., operatively connecting the longitudinal member 101 to the breakaway bar 201, or operatively connecting the breakaway bar 201 to the first elbow section 302, and/or operatively connecting the first elbow section 302 to the elongated member 301, and/or operatively connecting the elongated member 301 to the second elbow section 303, and/or operatively connecting the second elbow section 303 to the first handle bar 401, and/or operatively connecting the first handle bar 401 to the third elbow section 402, and/or operatively connecting the third elbow section 402 to the connector 403, and/or operatively connecting the connector 403 to the fourth elbow section 404, and/or operatively connecting the fourth elbow section 404 to the second handle bar 405, etc.).

Additionally the various angles provided by the elbow sections 302, 303, 402, or 404 assist in controlling the amount of force or torque required for the user (i.e., surgeon) to cause the implant portion 100 to breakaway from the inserter portion 300. When the breakaway bar 201 breaks, part or all of it may stay connected to the longitudinal member 101. Alternatively, when broken part or all of the breakaway bar 201 may stay connected to the first elbow section 302 of the inserter portion 300. The amount, if any, of the breakaway bar 201 which remains connected to any of the implant portion 100 and the inserter portion 300, respectively, depends on how the break occurs, where along the breakaway bar 201 the break occurs, the geometric configuration of the breakaway bar 201, the material properties of the breakaway bar and implant portion 100 and inserter portion 300, and/or a combination of these and other factors including the force and/or torque applied by the user (i.e., surgeon).

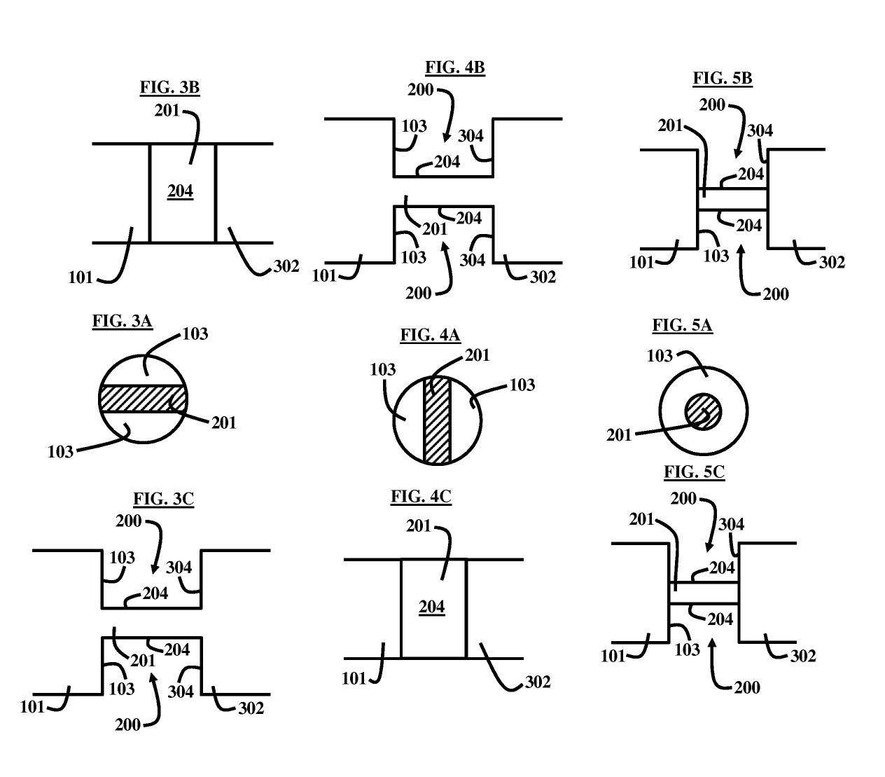

FIGS. 3A through 5C, with respect to FIGS. 1A through 2, and 6 illustrate example embodiments of the configuration of the breakaway bar 201 with respect to notch 200. For example, FIGS. 3A through 3C illustrate a substantially horizontally configured breakaway bar 201. FIGS. 4A through 4C illustrate a substantially vertically configured breakaway bar 201. FIGS. 5A through 5C illustrate a substantially round/cylindrical breakaway bar 201. Although not shown, the breakaway bar 201 could also include a tapered configuration such that the thickness/diameter of the breakaway bar 201 becomes progressively smaller the closer it gets to the second end 103 of the longitudinal member 101. This may assist in allowing a majority of the breakaway bar 201 to become detached from the longitudinal member 101 upon breakage. Furthermore, other configurations are possible in accordance with the embodiments herein, and the embodiments herein are not limited to any particular configuration.

Generally, the embodiments herein provide a notch 200 that permits the implant portion 100 to breakaway from a connected inserter portion 300 once the implant portion 100 is inserted into a construct assembly (not shown). In one embodiment, the implant portion 100 and the inserter portion 300 comprise a monolithic structure which is broken apart by breaking the breakaway bar 201 after the implant portion 100 has been properly inserted in a construct assembly (e.g., pedicle screw assembly) (not shown). Various handle positions and various geometric cuts in the notch 200 are possible in accordance with the embodiments herein.

Since the inserter portion 300 and the breakaway bar 201 take up no more space than the actual thickness/diameter of the longitudinal member 101, the device 50 enhances visibility, helps avoid error-prone user (i.e., surgeon) assembly of the implant portion 100 with the inserter portion 300 (since they are initially connected together during manufacturing), and alleviates the need for additional bulky attachments and components to the device 50. Accordingly, the embodiments herein improve MIS capabilities and provide a user-friendly technique of releasing the longitudinal member 101 in vivo once the construct assembly (e.g., pedicle screw assembly) (not shown) is assembled during the surgery.

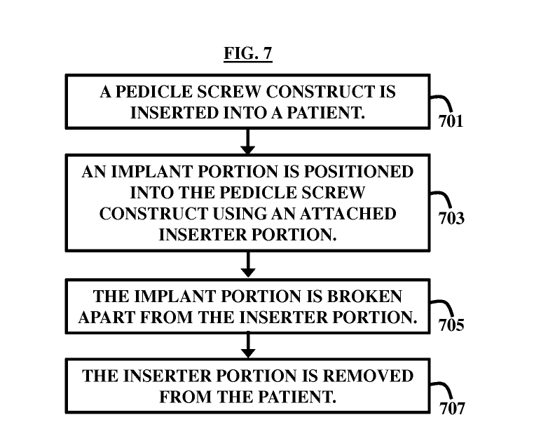

FIG. 7, with reference to FIGS. 1A through 6, illustrates a flow diagram according to an embodiment herein. First, a pedicle screw construct (not shown) (not including the rod) is inserted (701) into a patient. Next, an implant portion 100 is positioned (703) into the pedicle screw construct (not shown) using an attached inserter portion 300. Then, upon proper seating into the pedicle screw construct (not shown), the implant portion 100 is broken apart (705) from the inserter portion 300 by pulling, pushing, bending, and/or twisting, etc. the inserter portion 300 with respect to the implant portion 100, which causes the breakaway bar 201 to break thereby separating the implant portion 100 from the inserter portion 300. Finally, the inserter portion 300 is removed (707) from the patient and may be discarded.

Generally, the embodiments herein provide a medical device 50 comprising an implant portion 100, an inserter portion 300 detachably connected to the implant portion 100, and a device manipulation portion 400 operatively connected to the inserter portion 300. In one embodiment, the implant portion 100 comprises a longitudinal member 101. The device 50 may further comprise a breakaway component 201 in between the implant portion 100 and the inserter portion 300. The device 50 may further comprise a notch 200 configured between the implant portion 100 and the inserter portion 300. The implant portion 100 and the inserter portion 300 are detachable upon applying a sufficient force to the device manipulation portion 400 to cause detachment of the implant portion 100 from the inserter portion 300. The detachment of the implant portion 100 from the inserter portion 300 occurs after the implant portion 100 is seated in a construct assembly (not shown) that is attached to a bone (not shown) inside a patient (not shown).

The breakaway component 201 may be configured as a bar having a thickness less than or equal to a thickness of any of the implant portion 100 and the inserter portion 300. A portion of the breakaway component 201 remains attached to any of the implant portion 100 and the inserter portion 300 upon detachment of the implant portion 100 from the inserter portion 300. The device manipulation portion 400 may be configured as a handle. In one embodiment, the inserter portion 300 comprises a thickness less than or equal to a thickness of the implant portion 100. The notch 200 may be positioned on at least one side of the breakaway component 201. In one embodiment, the breakaway component 201 comprises a uniform thickness. In another embodiment, the breakaway component 201 comprises a tapered configuration. The device 50 may further comprise a first angled portion (e.g., elbow 302) in between the implant portion 100 and the inserter portion 300, and a second angled portion (e.g., elbow 303) in between the inserter portion 300 and the device manipulation portion 400. Additionally, the device 50 may further comprise at least one angled portion (e.g., elbows 402, 404) in the device manipulation portion 400.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A medical device comprising:
an implant portion;
an inserter portion detachably connected to said implant portion; and
a device manipulation portion operatively connected to said inserter portion.

2. The device of claim 1, wherein said implant portion comprises a longitudinal member.

3. The device of claim 1, further comprising a breakaway component in between said implant portion and said inserter portion.

4. The device of claim 3, further comprising a notch configured between said implant portion and said inserter portion.

5. The device of claim 3, wherein said implant portion and said inserter portion are detachable upon applying a sufficient force to said device manipulation portion to cause detachment of said implant portion from said inserter portion.

6. The device of claim 5, wherein said detachment of said implant portion from said inserter portion occurs after said implant portion is seated in a construct assembly that is attached to a bone.

7. The device of claim 3, wherein said breakaway component comprises a bar having a thickness less than or equal to a thickness of any of said implant portion and said inserter portion.

8. The device of claim 5, wherein a portion of said breakaway component remains attached to any of said implant portion and said inserter portion upon detachment of said implant portion from said inserter portion.

9. The device of claim 1, wherein said device manipulation portion comprises a handle.

10. The device of claim 1, wherein said inserter portion comprises a thickness less than or equal to a thickness of said implant portion.

11. The device of claim 4, wherein said notch is positioned on at least one side of said breakaway component.

12. The device of claim 3, wherein said breakaway component comprises a uniform thickness.

13. The device of claim 3, wherein said breakaway component comprises a tapered configuration.

14. The device of claim 1, further comprising:
a first angled portion in between said implant portion and said inserter portion; and
a second angled portion in between said inserter portion and said device manipulation portion.

15. The device of claim 1, further comprising at least one angled portion in said device manipulation portion.

16. A method of inserting an implant in a patient, said method comprising:
placing a pedicle screw construct assembly in said patient;
positioning an implant portion of a medical device into said pedicle screw construct assembly using an inserter portion that is detachably connected to said implant portion;
seating said implant portion into said pedicle screw construct assembly;
forceably detaching said implant portion from said inserter portion; and
removing said inserter portion from said patient.

17. The method of claim 16, further comprising manipulating said inserter portion with a handle.

18. The method of claim 17, wherein said implant portion and said inserter portion are detachable upon applying a sufficient force to said handle to cause detachment of said implant portion from said inserter portion.

19. The method of claim 16, wherein said forceably detaching said implant portion from said inserter portion comprises any of pulling, pushing, bending, and twisting said inserter portion with respect to said implant portion.

20. The method of claim 16, further comprising discarding said insertion portion after removing said inserter portion from said patient.
